(19) United States
(12) Patent Application Publication
(10) Pub. No.: US 2022/0088404 A1
(43) Pub. Date: Mar. 24, 2022

(54) Methods and System for Modeling Electromagnetic Brain Stimulation and Brain Recordings with Boundary Element Approach with Fast Multipole Acceleration

(71) Applicant: Neva Electromagnetics, LLC, Yarmouth Port, MA (US)

(72) Inventor: Sergey N. Makarov, Holden, MA (US)

(21) Appl. No.: 17/029,679

(22) Filed: Sep. 23, 2020

(51) Int. Cl. A61N 2/02; A61B 5/0484; A61B 5/00; A61N 2/00; A61N 1/36; A61N 1/05

(52) U.S. Cl. CPC A61N 2/02; A61B 5/0484; A61B 5/7242; A61N 1/0534; A61N 1/3606; A61N 1/36135; A61N 2/006

(57) ABSTRACT

The present invention relates bringing a new accessible and accurate computational modeling approach to compute electric and magnetic fields in the brain to clinical practice.

### FIELD OF THE INVENTION

The present invention relates bringing a new accessible and accurate computational modeling approach to compute electric and magnetic fields in the brain to clinical practice.

### BACKGROUND OF THE INVENTION

The solution of computational electromagnetics (CEM) problems for human brain is of much interest to medical as well as commercial applications. Efficient and accurate CEM simulation enables, for example, a clinician to estimate and determine the exact locations of natural neural activity in the cortex or optimize the dose and the positions of electrodes or coils for electric or magnetic brain stimulation, providing in many cases more information than can ever be measured in a laboratory or in situ.

Commonly used numerical techniques for the analysis of electrical and magnetic brain activity as swell as electrical and magnetic brain stimulation are

a) Finite Element Method (FEM);

b) Boundary Element Method (BEM);

Bioelectromagnetic modeling of the brain as well as the human body is a continued competition between FEM and BEM. FEM discretizes the entire 3D volume into M tetrahedra or hexahedra. However, the resulting M×M system matrix is sparse. Its filling and iterative solution require as low as O(M log M) operations. BEM only discretizes 2D boundaries between otherwise homogeneous tissues into N triangles or quadrilaterals. However, the resulting N×N system matrix is dense; its filling alone requires O(N²) operations. Although N<<M, FEM is winning for large M and N i.e. for high-resolution subject-specific head models. For this reason, FEM is presently dominant in modeling chief neuromodulation modalities: transcranial magnetic stimulation, transcranial electrical stimulation, and deep brain stimulation. However, FEM cannot efficiently handle singular point sources—EEG (electroencephalography) and MEG (magnetoencephalography) dipoles. Therefore, a low-resolution BEM is used in EEG/MEG modeling.

The fast multipole method (FMM) introduced by Rokhlin and Greengard in 1980s is a way to reduce O(N²) to O(N log N) BEM operations and restore the major advantage of BEM—its faster speed and better accuracy for piecewise homogeneous tissues.

Since the 2000s, the high-frequency BEM-FMM algorithm has been successfully applied to the modeling of electromagnetic and acoustic scattering and radiation problems with a focus on defense applications. The quasistatic BEM-FMM algorithm has also been applied to capacitance/inductance computations in electrical engineering. Several commercial engineering software packages use custom BEM-FMM solvers, including ANSYS Q3D Extractor and FEKO.

However, before our first publication (Makarov et al. A Quasi-Static Boundary Element Approach with Fast Multipole Acceleration for High-Resolution Bioelectromagnetic Models. IEEE Trans Biomed Eng. 2018 Mar. 7. doi: 10.1109/TBME.2018.2813261) there was only one attempt to employ the fast multipole method (FMM) to quasistatic bioelectromagnetic problems (Kybic et al Fast multipole acceleration of the MEG/EEG boundary element method. Physics in Medicine and Biology. 2005; 50(19), 4695-4710. doi: http://doi.org/10.1088/0031-9155/50/19/018). This publication predicted that the time complexity of FMM may be higher rather than lower than that of traditional methods. We fear that this interpretation has had a discouraging influence on the development of approaches employing FMM in brain research.

Preliminary work done by the authors over 2018-2020 indicates that the meaningful multipole acceleration and the best combined boundary element fast multipole method (B EM-FMM) is obtained when
i. Surface-charge formulation of the integral equation is used for the BEM method;
ii. The general-purpose widely-known and proven FMM algorithm such as https://github.com/flatironinstitute/FMM3D, hereinafter, FMM3D is used to accelerate BEM and compute quasistatic brain fields in a straightforward, efficient, simple, and reproducible way.

The BEM-FMM approach so developed is in a position to provide
i. High speed and accuracy exceeding that of the comparable finite element method (FEM) for piecewise-isotropic high-resolution problems.
ii. Unconstrained numerical field resolution close to cortical surfaces. This is not limited by the FEM volumetric mesh size and can reach a micron scale if desired.
iii. Inherent ability to model internal singular sources such as EEG/MEG dipoles as opposed to FEM.
iv. Ability to model fields and sources at multiscale and within dense realistic neuronal arbor.

At the same time, an implementation of the BEM-FMM approach for practical brain modeling problem including both neurophysiological recordings and neurostimulation will require:
i. The method for inclusion and modeling non-nested (non-onion) head topologies which are increasingly more frequently employed for high-resolution simulation tasks. The standard BEM used previously and formulated in terms of the electric potential cannot s accomplish this task.
ii. The method for inclusion and modeling anisotropic tissues such as skull or white matter. The standard BEM cannot accomplish this task.
iii. The method for modeling external (scalp) and internal (intracortical microstimulation) voltage electrodes.
iv. The method for inclusion and modeling external (scalp) and internal (intracortical microstimulation) current electrodes.
v. The method for improving the convergence of the BEM-FMM iterative solution using the charge conservation law.
vi. The method for restoring near-surface normal electric fields from the computed charge distribution without postprocessing and in no time.
   These six methods are the subject of the present invention.

### SUMMARY OF THE INVENTION

Methods and system are presented for making the new BEM-FMM computational approach formulated in (Makarov et al. IEEE Trans Biomed Eng. 2018. doi: 10.1109/TBME.2018.2813261, Makarov et al J Neural Eng. 2020 doi: 10.1088/1741-2552/ab85b3, Makarov et al. IEEE Trans. Biomed. Eng. 2020 doi: 10.1109/TBME.2020.2999271) applicable to practical brain modeling problems in clinical applications including neurostimulation (TES or transcranial electrical stimulation, DBS or deep brain stimulation, ICMS or intracortical microstimulation) and neurophysiological recordings (EEG, MEG, intracortical EEG or iEEG) and. Examples of those applications are shown in FIG. 1. All applications require fast and accurate computations of the electric field and the electric potential within the head, which can be performed with BEM-FMM. Neurophysiological recordings additionally include magnetic field measurements (MEG, not shown in FIG. 1).

These methods include:
i. The method for inclusion and modeling non-nested (non-onion) head topologies which are increasingly more frequently employed for high-resolution simulation tasks. The standard BEM used previously and formulated in terms of the electric potential cannot accomplish this task.
ii. The method for inclusion and modeling anisotropic tissues such as skull or white matter. The standard BEM cannot accomplish this task.
iii. The method for modeling external (scalp) and internal (intracortical microstimulation) voltage electrodes.
iv. The method for inclusion and modeling external (scalp) and internal (intracortical microstimulation) current electrodes.
v. The method for improving the convergence of the BEM-FMM iterative solution using the charge conservation law.
vi. The method for restoring near-surface normal electric fields from the computed charge distribution without postprocessing and in no time.

In a preferred embodiment, these methods will be used coupled with the new BEM-FMM approach. This approach includes the integral equation in terms of surface charge density on conductivity boundaries and the integration of the open-source parallelized FMM library, FMM3D originating from the inventors of the FMM and made accessible for commercial use since 2017 (Gimbutas et al. fmm3D Documentation. Release 0.1.0. 2019. Online: https://github.com/flatironinstitute/FMM3D). This FMM3D library summarizes a more than 20 years long ongoing effort of a group of leading mathematicians. This FMM3D library possesses high performance and speed, which could hardly be surpassed by custom codes.

In a preferred embodiment, these methods will assess brain waves and brain stimulation within a system on a computer, providing more information than can ever be measured in a laboratory or in situ.

### DETAILED DESCRIPTION OF THE INVENTION

I Overview

Methods and system are presented for assessing electric and magnetic quasistatic brain waves (EEG/MEG/iEEG) due to neural activity as well as assessing brain stimulation (TES/DBS/ICMS) fields on a computer. This is done while improving speed and accuracy of computational electromagnetics modeling environments. For the purpose of clarity, several embodiments described below are presented in the context of surface head models, using triangular surface discretization. But those of skill in the art will recognize that the system and methods described herein below are applicable to alternative discretization approaches (e.g., rectangular, hexagonal lattice, etc.). In addition, those of skilled in the art will recognize that the principles can also be applied to determining performance characteristics for human head models that include dielectric tissue properties.

In some embodiments, the system and methods are embodied in software stored and executable on one or more computing devices. A user can access the systems and methods using a user device, e.g., a computing device, remotely or locally. For example, the user device can interact with the systems and methods through a network, e.g., the Internet, and can display a user interface, e.g., a webpage, to the user. The user device can also host the systems and methods locally, e.g., by downloading and installing the software on the user device. The models can be constructed using the same systems and methods or different systems and methods that are hosted by the same computing device(s).

II. Example of System

In the example shown in FIG. 2, a client device 110 accesses and interacts with a server device 150 through a network 140 to obtain information about scattering/radiation of a target or choose a target model for use. A user may interact with the client device 110 directly or through one or more input devices 120. The input device(s) 120 can be part of the client device 110 and can be a user device. In some implementations, the client device 110 can provide the desired information without needing to access the server device 150. In some implementations, historical data is provided to the server device 150 or to the client device 110 to generate and process the target models. In some implementations, the server device 150 or the client device 110 does not generate target models. Instead, target models are provided to the server device 150.

The server device 150 hosts a technical computing environment (TCE) 130 in which the historical data is processed and the target models are processed and/or generated. In some implementations, the client device 110 may employ a TCE 130 (160) to provide a display, e.g., a webpage. In some implementations, a user interacts with the server device 150 via the displayed webpage. The displayed TCE 130 operating on the client device 110 can be the same as or be part of the TCE 130 hosted by the server device 150. In some implementations, the TCE 160 is hosted on the client device 110. In such implementations, the client device 110 may not need to access the server device 150 to generate and/or process the desired model. Instead, target models can be generated locally at the client device 110 in the TCE 130. The TCE 130 can provide an interface that allows a user to interact with the client device 110 and the server device 150.

The server device 150, the client device 110, and the network 140 can be connected through wired connections, wireless connections, or a combination of wired and wireless connections. Although one client device 110 and one server device 150 are shown, multiple client devices 110 can access one or more server devices 150 through the same network 140 or a different network. In some implementations, one or more server devices 150 host one or more TCEs 130, e.g., using virtual machines. Multiple server devices 150 can be used to execute program code, e.g., serially or in parallel, and may provide respective results of executing the program code to the client device 110.

The client device 110 can include one or more devices capable of receiving, generating, storing, processing, and/or providing program code and/or information associated with program code for a model. For example, the client device 110 includes a computing device, such as a desktop computer, a laptop computer, a tablet computer, a mobile phone (e.g., a smart phone, a radiotelephone, etc.), or a similar device. In some implementations, the client device 110 receives information from and/or transmits information to the server device 150.

The server device 150 may include one or more devices capable of receiving, generating, storing, processing, evaluating, and/or providing program code and/or information associated with program code for a model. Additionally, the server device 150 also includes one or more devices capable of comparing multiple models generated from the same set of historical data. For example, the server device 150 includes a computing device, such as a server, a desktop computer, a laptop computer, a tablet computer, or a similar device.

The network 140 can include one or more wired and/or wireless networks. For example, network 140 includes a cellular network, a public land mobile network (PLMN), a local area network (LAN), a wide area network (WAN), a metropolitan area network (MAN), a telephone network (e.g., the Public Switched Telephone Network (PSTN)), an ad hoc network, an intranet, the Internet, a fiber optic-based network, a private network, a cloud computing network, and/or a combination of these or other types of networks.

Referring also to FIG. 3, the TCE 130/160 is supported by an application programming interface (API) 260. The API 260 can be visible to a user within the TCE 130, or can be invisible to the user. In some implementations, the API 260 specifies how portions of programming code implementing different parts of the methods of this disclosure, e.g., generating and/or comparing infinite array models, interact with each other to perform the methods and generate results. An example of a portion of programming code is an optimizer 266 that is capable of optimizing parameters. Other examples of the programming code portions can include, but not limited to, a target model generator, equation solver, and graphical user interface (not shown). The API 260 can receive one or more TCE inputs 262 from the TCE 130, or on behalf of a user or a user device, and output one or more TCE outputs 264 to the TCE 130. Examples of TCE inputs 262 include, for example, specifications for the target models to be processed or generated, incident field data, testing data, etc. Examples of TCE outputs 264 include generated models, results of model comparisons, and other user specified information.

The numbers and arrangements of devices and networks shown in FIGS. 2 and 3 are provided as examples. In practice, there can be additional devices and/or networks, fewer devices and/or networks, different devices and/or networks, or differently arranged devices and/or networks than those shown in the figures. Furthermore, two or more devices shown in the figures can be implemented within a single device, or a single device shown in the figures can be implemented as multiple, distributed devices.

III. Example Implementation of Brain Modeling with Voltage/Current Active or Passive Electrodes Using Non-Nested Head Topology and Tissue Anisotropy

With reference to FIG. 4, the system of this disclosure, including software stored on the server device 150, the client device 110, TCEs 130/160 of FIGS. 2 and 3, can perform process 300 to acquire human head model of the subject of interest, and solve for the associated electric/magnetic fields and other results. Although a particular sequence is described for the process 300, skilled artisans will appreciate that alternative mathematical approaches may be possible for particular steps. Further it may be possible to perform one or more steps in the process 300 at a different time relative to the other steps and/or in orders other than as shown in FIG. 3.

Initially, upon receipt of a request, step 302 to perform brain modeling e.g., from a user or a user device, the system may prompt the user or user device to input complete information about the target model(s), step 304. These inputs can be the same as TCE inputs 262 of FIG. 3. For example, the systems may ask the user to import the surface head mesh of a subject in the form of *.stl files and provide input on one or more of the following parameters: conductivity values for head compartments, dielectric constant values for head compartments, electrode geometry, sizes, positions, spatial points of interest for electric and magnetic fields determination, and desired TCE outputs 264 to be generated and/or or displayed. The systems may present, e.g., to the user, choices for options, for one or more of these inputs for selection. In some implementations, the systems interact with the user or the user device dynamically, e.g., in multiple steps, to prompt the user or the user device to provide the required or the missing inputs. The systems may not need to generate the target model from input requirements. Instead, the systems may receive the target model from an external input, e.g., from the user or the user device. When some inputs are not received, the systems may use one or more default parameters and inform the user that a default parameter has been used.

Once the target surface head model is imported and described, some implementations then begin to model the fields within the head and performance estimation process (Step 308). Mathematical details of certain exemplary implementations of process 308 will be described in much greater detail below as including numerous sub-processes. In step 306, the target head surface mesh model is also processed internally to enable a proper treatment of the non-nested head geometry. FIG. 5 illustrates the algorithm, which is described in great detail below in the text. In Step 310, a uniform dipole mesh is introduced into an anisotropic head compartment as shown in FIG. 6. In Step 312, the electrodes with known parameters are either imprinted or embedded automatically and their mesh labels are generated. In Step 314, neighbor potential integrals necessary for accurate integration are computed as described below. In steps 316, 318, 320, 322, 324, the resulting system of BEM equations is solved iteratively using fast multipole method (FMM) acceleration and generalized minimum residual method (GMRES).

Results, such as described above, of the electric and magnetic field estimation process 308 may be output in Step 330. A determination (Step 332) may then be made to establish whether the determined performance characteristics of the electrode system and its readings meet user's requirements.

Typical performance requirements specified for neurostimulation (TMS/DBS/ICMS) include sufficiently high electric field levels within certain cortical and subcortical domains and sufficiently low fields otherwise.

Typical performance requirements specified for neurophysiological recordings include sufficiently high output voltage signals at the electrodes and sufficient electrode coverage for required spatial resolution.

If the requirements are satisfied, the process may then proceed to other target modeling tasks, or progress to prototype development (Step 334). If the performance measures output does not meet the specified requirements, the process may iterate, e.g., from step 332, permitting the user to enter additional and/or different input parameters or change the electrode assembly, or the system to automatically vary certain input parameters, until the output performance characteristics for the modeled physical field distributions satisfy the requirements.

Sections IV through XII will now describe in detail exemplary solutions for the scattering algorithm with the general purpose fast multipole method, FMM3D, of process 300 including step 308 and individual steps 306, 310, 312, 314, 316, 318, 320, 322, 324.

IV. Direct Charge-Based BEM in a Conducting Medium—Relevant to Step 308 and all Individual Steps 306, 310, 312, 314, 316, 318, 320, 322, 324

Induced charges with a surface charge density ρ(r) in C/m² reside on macroscopic or microscopic tissue conductivity interface(s) S once an external electromagnetic stimulus (a primary electric field E^{p}(r), either conservative or solenoidal) is applied. The induced surface charges alter (typically block and/or redirect) the primary stimulus field. The total electric field anywhere in space except the charged interfaces themselves is governed by Coulomb's law

$$E ( r ) = {E}^{p} ( r ) + {E}^{s} ( r ) = {E}^{p} ( r ) + {\int }_{S} \frac{ρ ( {r}^{′} )}{4 π {ɛ}_{0}} \frac{r - {r}^{′}}{{\vert r - {r}^{′} \vert}^{3}} {dr}^{′} , r ∉ S \qquad ( Eq .1 )$$

where ε₀ is dielectric permittivity of vacuum. The electric field is discontinuous at the interfaces. When approaching a charged interface S with a normal vector n from either direction (inside or outside with regard to the direction of the normal vector), the electric field is given by

$${E}_{i n / o ut} = {E}^{p} + {\int }_{S} \frac{1}{4 π {ɛ}_{0}} \frac{r - {r}^{′}}{{\vert r - {r}^{′} \vert}^{3}} ρ ( {r}^{′} ) {dr}^{′} ∓ n ( r ) \frac{ρ ( r )}{2 {ɛ}_{0}} , r ∈ S \qquad ( Eq .2 )$$

An integral equation for ρ(r), is obtained after substitution of Eq. (2) into the quasistatic boundary condition, which enforces the continuity of the normal current component across the interface, that is

σᵢₙn(r)·Eᵢₙ=σₒᵤₜn(r)·Eₒᵤₜ, r∈S (Eq. 3)

The result has the form of the Fredholm integral equation of the second kind.

$$\frac{ρ ( r )}{2} - K n ( r ) \cdot {\int }_{S} \frac{1}{4 π} \frac{r - {r}^{′}}{{\vert r - {r}^{′} \vert}^{3}} ρ ( {r}^{′} ) {dr}^{′} = K n ( r ) \cdot {ɛ}_{0} {E}^{p} ( r ) , r ∈ S \qquad ( Eq .4 )$$

where the electric conductivity contrast

$$K = \frac{{σ}_{i n} - {σ}_{o u t}}{{σ}_{i n} + {σ}_{o u t}}$$

is defined at the interface(s). Here, σᵢₙ, σₒᵤₜ are are the conductivities inside and outside with regard to the direction of the normal vector, respectively. Note that if we solve Eq. (4) and then substitute the result for ρ(r) in Eq. (1), the normalization constant ε₀ will cancel out. Therefore, its exact value does not matter for the subsequent analysis. However, if the displacement currents are significant, extra bound polarization charges will reside on the interfaces. Their effect is taken into account by considering a complex conductivity in the form a σ→σ+jωε for a harmonic excitation with angular frequency co in Eq. (4).

The surface charge density is expanded/discretized into pulse bases (zeroth-order basis functions) on triangular facets tₘ with area Aₘ. The surface charge density is thus constant for every facet. The Galerkin method is then applied to Eq. (4) which gives us a system of M linear equations for unknown expansion coefficients cₘ in the form

$$\frac{{c}_{m}}{2} - \frac{K}{{A}_{m}} {\sum }_{n = 1}^{M} ( {n}_{m} \cdot \int {\int }_{{A}_{m} {A}_{n}} \frac{1}{4 π} \frac{r - {r}^{′}}{{\vert r - {r}^{′} \vert}^{3}} {dr}^{′} dr ) {c}_{n} = \frac{K}{{A}_{m}} {ɛ}_{0} {\int }_{{A}_{m}} {n}_{m} \cdot {E}^{p} ( r ) dr , m = 1 : M \qquad ( Eq .5 )$$

The double potential integrals present in Eq. (5) require care in their numerical evaluation. Facets which are spatially close to one another (i.e., not considered well separated on the lowest level of the FMM octree) cannot be treated with the FMM. The close facets are those whose center-to-center distances are on the order (1-10 times) of a linear face size computed, for high-quality surface meshes, as the square root of the face area. The corresponding nearfield potential integrals are instead directly calculated and stored in the sparse nearfield BEM matrix using analytical integration for the inner integral and a Gaussian quadrature of 10th degree of accuracy for the outer integrals. The number of geometrical (based on Euclidian distance) neighbors in Eq. (5) may vary, but a relatively small number may be adequate. It must be noted that these geometrical neighbors may belong to different tissue compartments.

V. Application of General-Purpose Fast Multipole Method—Relevant to Step 308 and all Individual Steps 306, 310, 312, 314, 316, 318, 320, 322, 324

The general-purpose FMM and its most recent freeware distribution (Gimbutas et al. fmm3D Documentation. Release 0.1.0. 2019) is applied to compute all integrals of type defined by Eq. (5) except for neighbor integrals, using the center-point approximation at face centers rₘ, yielding

$${\int }_{{t}_{m}} {\int }_{{t}_{n}} \frac{r - {r}^{′}}{{\vert r - {r}^{′} \vert}^{3}} {drdr}^{′} \approx {A}_{m} {A}_{n} \frac{{r}_{m} - {r}_{n}}{{\vert {r}_{m} - {r}_{n} \vert}^{3}} \qquad ( Eq .6 )$$

This problem is equivalent to finding the electric field at target points rₘ generated by the point charges located at source points rₙ. The accuracy of the FMM (the number of levels) is conventionally estimated for arbitrary volumetric charge distributions. However, for surface-based charge distributions, a much better relative accuracy is observed. For example, with the intrinsic method accuracy set as ε=0.1, the mean error for the pial cortical surface (GM shell) may be as low as 0.1% with respect to the electric field amplitude and 0.08 deg with respect to the field angle deviation as compared to the most accurate solution (i.e., the solution where FMM precision is set to maximum).

VI. The Method for Inclusion and Modeling Non-Nested (Non-Onion) Head Topology—Step 306

For the onion-like models, there should be none of coincident surfaces, because tissues of these models surround and enclose each other without touching—they are hollow shells, where each shell segments a boundary between exactly two tissue types.

For the non-nested models (an example is the MIDA model, Iacono et al, PLoS One 2015; 10(4). doi: 10.1371/journal.pone.0124126) however, the interior and exterior boundaries of every tissue are explicitly segmented. This means, for example, that the MIDA model's white matter and gray matter both independently segment their mutual boundary, producing a large number of duplicate facets. These duplicate facets would produce singularities that invalidate simulation results, so they are resolved as described below. The BEM-FMM approach theoretically allows to handle non-nested geometries since it uses only the local boundary condition Eq. (3) and does not uses integral transformations for manifold or closed surfaces like the second Green's identity. The practical method for doing so is proposed and tested as stated below.

If the surface is a 2-manifold object with no contact to other surfaces (a “nested” topology where each of the surfaces is associated with a single unique exterior compartment), n is simply the outer normal vector to the surface; σᵢₙ is the conductivity inside the object; and σₒᵤₜ is the conductivity of the surrounding medium. If two objects (1 and 2) are in contact with each other as shown in FIG. 5, the joint interface between them should be counted only once. In FIG. 5, this interface is counted only for object 1 with σ₁ being the inner conductivity and σ₂ being the outer conductivity (in the direction of the normal vector n₁). Facets of object 2 at the interface are now removed to avoid double-counting. Alternatively, the interface may belong only to object 2, with the direction of the normal vector and the conductivity values switched. In that case, facets of object 1 at the interface would be removed. From the formal point of view, one needs a composite mesh without double coincident facets. Then, for an arbitrary triangular facet tₘ of the mesh with a given unit normal vector nₘ, one needs to know the conductivity σ_{m,out} “outside” (i.e. in the direction of nₘ) and the conductivity σ_{m,in} “inside” (i.e. in the opposite direction of nₘ). This information is sufficient to completely describe the model.

The practical realization is as follows. For each pair of duplicate facets, one is designated the facet to be deleted, and the other is designated the facet to be kept. The outer conductivity of the facet to be kept is set equal to the inner conductivity of the facet to be deleted, and associated conductivity contrast information is updated for the facet to be kept. The facet to be deleted, and all associated information, is then removed from the model. FIG. 5 quantifies the results of this operation. The method was tested by comparison with proven FEM software and demonstrated excellent accuracy.

VII. The Method for Inclusion and Modeling Anisotropic Tissues Such as Skull or White Matter—Step 310

An anisotropic medium can be treated as a model of a realistic inhomogeneous medium with specifically oriented microinclusions: an example is given by white matter fiber. When an external electric field is applied, the microinclusions become polarized. This polarization is of a dipole nature—one part of the inclusion becomes positively charged while another part is charged negatively, with the total charge equal to zero. Therefore, we can replace the microinclusions by secondary dipoles densely and uniformly distributed in a volume. Those should be field-dependent dipoles, similar to the linear dependent sources in electrical engineering. When the external electric field is absent their strength (the dipole moment) will be equal to zero. Otherwise, the dipole moment will be proportional to the field. Three degrees of freedom (x-, y- and z-dipoles) should be made available at any spatial point to account for three local electric-field components as shown in FIG. 5.

The strength of dependent dipole sources, say, in the x-direction is made proportional to (σₓ−σ_{ref})/(σₓ+σ_{ref}) where σₓ is the component of the conductivity tensor in the x-direction, and σ_{ref} is a reference conductivity. The reference conductivity maybe chosen as the largest component of the conductivity tensor.

The BEM-FMM is the preferred platform for the implementation since the number of dependent sources is practically unlimited if the fast multipole method is used to compute the combined effect of their electric fields.

The resulting system of integral equations can be solved iteratively, by updating the field of all dependent sources at every iteration step, and then computing the medium feedback due to this field. The method convergence is excellent.

This approach has been tested by comparison with a high-end commercial FEM software ANSYS Maxwell with adaptive mesh refinement. For highly anisotropic specimens with the top surface electrodes, it generates relative field/electric current errors below 5-10% in the entire volume for the uniformly-distributed dipole grid.

VIII. The Method for Modeling External (Scalp) and Internal (Intracortical Microstimulation) Voltage Electrodes—Steps 312, 316, 318

In TES/DBS/ICMS problems, the primary electric field E^{p}(r) in Eq. (4) is zero. However, voltage V is impressed at certain electrode surfaces Sₑ. Voltage V may have different values for different electrodes. The electrode surfaces are considered as being a part of other closed surfaces, for example a part of the insulated lead surface. At the electrode surfaces, the Fredholm integral equation of the first kind is imposed instead of Eq. (4), that is

$${\int }_{{S}_{e}} \frac{ρ ( {r}^{′} )}{4 π {ɛ}_{0} \vert r - {r}^{′} \vert} {dr}^{′} = V \qquad ( Eq .7 )$$

Furthermore, the conductivity contrast value is set to one at electrodes which corresponds to a very high metal (copper, platinum, or iridium) conductivity as compared to the conductivity of the surrounding tissues.

The surface charge density is expanded/discretized into pulse bases (zeroth-order basis functions) on triangular facets tₘ with area Aₘ. The Galerkin method is then applied to Eq. (4) and Eq. (7), which gives a system of M linear equations for unknown expansion coefficients cₘ in the form

$$\frac{{c}_{m}}{2} - \frac{K}{{A}_{m}} {\sum }_{n = 1}^{M} ( {n}_{m} \cdot \int {\int }_{{A}_{m} {A}_{n}} \frac{1}{4 π} \frac{r - {r}^{′}}{{\vert r - {r}^{′} \vert}^{3}} {dr}^{′} dr ) {c}_{n} = \frac{K}{{A}_{m}} {ɛ}_{0} {\int }_{{A}_{m}} {E}^{p} ( r ) dr , m = 1 : M \qquad ( Eq .8 a )$$

and N remaining linear equations for unknowns cₘ on the electrode surface in the form

$${g}_{m} {\sum }_{n = 1}^{N} ( \int {\int }_{{A}_{m} {A}_{n}} \frac{1}{4 π {ɛ}_{0}} \frac{1}{\vert r - {r}^{′} \vert} {dr}^{′} dr ) {c}_{n} = {g}_{m} {\int }_{{A}_{m}} V d r , m = 1 : N \qquad ( Eq .8 b )$$

Coupled Eqs. (8a) and (8b) are solved simultaneously using a proper weighting gₘ. Without this weighting, an iterative solution will diverge. The established weighting is as follows

$${g}_{m} = {[ \int {\int }_{{A}_{m} {A}_{m}} \frac{1}{4 π {ɛ}_{0}} \frac{1}{\vert r - {r}^{′} \vert} {dr}^{′} dr ]}^{- 1} \qquad ( Eq .9 )$$

This weighting is equivalent to the proper diagonal preconditioner for Eq. (8b).

As already described in [0034], the general-purpose FMM and its most recent freeware distribution (Gimbutas et al. fmm3D Documentation. Release 0.1.0. 2019) is applied to compute all double integrals of type defined by Eq. (8a) except for neighbor integrals. The same method is now applied to compute all double integrals of type defined by Eq. (8b) except for neighbor integrals using the center-point approximation at face centers rₘ, yielding

$${\int }_{{t}_{m}} {\int }_{{t}_{n}} \frac{1}{\vert r - {r}^{′} \vert} {drdr}^{′} \approx {A}_{m} {A}_{n} \frac{1}{\vert {r}_{m} - {r}_{n} \vert} \qquad ( Eq .10 )$$

This problem is equivalent to finding the electric field at target points rₘ generated by the point charges located at source points rₙ. The accuracy of the FMM (the number of levels) is conventionally estimated for arbitrary volumetric charge distributions. However, for surface-based charge distributions, a much better relative accuracy is observed. For example, with the intrinsic method accuracy set as ε=0.1, the mean error for the pial cortical surface (GM shell) may be as low as 0.1% with respect to the electric field amplitude and 0.08 deg with respect to the field angle deviation as compared to the most accurate solution (i.e., the solution where FMM precision is set to maximum).

IX. The Method for Modeling External (Scalp) and Internal (Intracortical Microstimulation) Current Electrodes—Steps 312, 322, 324

The primary electric field E^{p}(r) in Eq. (4) is non-zero. It is impressed at certain electrode surfaces Sₑ in the form of the impressed current Iₑ. The BEM-FMM solution of Eq. (4) is obtained with the primary field n(r)·E^{p}(r)=±Iₑ/(σₑAₑ) impressed at the surfaces of the electrode and the reference electrode, respectively. Here, Aₑ is the electrode area and σₑ is the conductivity of the medium enclosing the electrode. For on-scalp electrodes, σₑ is the scalp conductivity.

X. Computing Neighbor Potential Integrals—Step 314

The double potential integrals present in Eqs. (8a) and (8b) require care. Facets which are spatially close to one another (i.e., not considered well separated on the lowest level of the FMM octree) cannot be treated with the FMM. These nearfield potential integrals are instead directly calculated and stored in the sparse nearfield BEM matrix using analytical integration for the inner integral and a Gaussian quadrature of 10^{th} degree of accuracy for the outer integrals. The number of geometrical (based on Euclidian distance) neighbors in Eqs. (7) may vary, but a relatively small number (4-64) may be adequate. It must be noted that these geometrical neighbors may belong to different tissues.

XI. The Method for Improving the Convergence of the Iterative Solution Using the Charge Conservation Law—Steps 318, 324

The charge conservation law is not explicitly included in Eq. (4); it could be enforced in the form

∫_{S}p(r′)dr′=0 (Eq. 11)

In Eq. (11), S is now the combination of all interfaces. Proper implementation of Eq. (11) implies a direct summation of integral on the right-hand side of Eq. (11) with Eq. (4) with a certain weighting; it provides a significantly better and unconstrained convergence rate of the iterative solution as shown in FIG. 7.

XII. The Method for Restoring Near-Surface Normal Electric Fields from the Computed Charge Distribution without Postprocessing and in No Time—Step 330

A significant advantage of the BEM-FMM approach established below is an ability to precisely obtain electric fields normal to the cortical surfaces (or any other interfaces) without additional computational cost or postprocessing. Only the solution for the surface charge density is necessary. After taking the scalar product of Eq. (2) with the surface normal vector n, Eq. (4) may then be substituted to explicitly find the normal electric field just inside the surface, n·Eᵢₙ(r); the normal electric field just outside the surface, n·Eₒᵤₜ(r); and the normal field discontinuity for any conducting interface, dn·E. All three quantities are directly proportional to each other. One has, for any conducting interface S and any observation point r∈S,

$$n \cdot {E}_{i n} ( r ) = \frac{{σ}_{out}}{{σ}_{i n} - {σ}_{out}} \frac{ρ ( r )}{{ɛ}_{0}} , n \cdot {E}_{out} ( r ) = \frac{{σ}_{i n}}{{σ}_{i n} - {σ}_{out}} \frac{ρ ( r )}{{ɛ}_{0}} , dn \cdot E = \frac{ρ ( r )}{{ɛ}_{0}} \qquad ( Eq .12 )$$

We should note that the normal component of the total E-field just inside/outside conductivity boundaries depends explicitly only on the induced surface charge density. For example, Eq. (12) immediately predicts that the normal component of the field just inside the outermost skin surface is always zero (there is indeed no current into air) since σₒᵤₜ is equal to zero in the first expression of Eq. (12). However, both the surface charge density and the normal component of the field just outside the outermost surface are certainly different from zero.

As used herein in relation to computing, the term component is intended to be broadly construed as hardware, firmware, and/or a combination of hardware and software.

Program code, referred to herein as code or program code, is to be broadly interpreted to include text-based code that may not require further processing to execute (e.g., C or C++ code, Hardware Description Language (HDL) code, very-high-speed integrated circuits (VHSIC) HDL (VHDL) code, Verilog code, Java code, another type of hardware and/or software based code that may be compiled and/or synthesized, etc.), binary code that may be executed (e.g., executable files that may be directly executed by an operating system, bitstream files that may be used to configure an FPGA, Java byte code, object files combined together with linker directives, source code, makefiles, etc.), text files that may be executed in conjunction with other executables (e.g., Python text files, Octave files, MATLAB files, a collection of dynamic-link library (DLL) files with text-based combining, configuration information that connects pre-compiled modules, an extensible markup language (XML) file describing module linkage, etc.), graphical design models that may be executed in conjunction with other executables (e.g., LabView models, SystemVue models, Simulink models, Ptolemy models), source code (e.g., readable by a human), machine code (e.g., readable by a machine), or the like. In some implementations, program code may include different combinations of the above-identified classes of code (e.g., text-based code, graphical models, binary code, text files, source code, machine code, etc.). Additionally, or alternatively, program code may include code generated using a dynamically-typed programming language (e.g., the M language, a MATLAB® language, etc.) that may be used to express problems and/or solutions using mathematical notations. Additionally, or alternatively, program code may be of any type, such as a function, a script, an object, etc.

It will be apparent that system and/or methods, described herein, may be implemented in different forms of hardware, firmware, or a combination of hardware and software. The actual specialized control hardware or software code used to implement these systems and/or methods is not limiting of the implementations. Thus, the operation and behavior of the system and/or methods were described herein being understood that software and hardware can be designed to implement the systems and/or methods based on the description herein.

Even though particular combinations of features are recited in the claims and/or disclosed in the specification, these combinations are not intended to limit the disclosure of possible implementations. In fact, many of these features may be combined in ways not specifically recited in the claims and/or disclosed in the specification. Although each dependent claim listed below may directly depend on only one claim, the disclosure of possible implementations includes each dependent claim in combination with every other claim in the claim set.

No element, act, or instruction used herein should be construed as critical or essential unless explicitly described as such. Also, as used herein, the articles “a” and “an” are intended to include one or more items, and may be used interchangeably with “one or more.” Furthermore as used herein, the term “set” is intended to include one or more items, and may be used interchangeably with “one or more.” Where only one item is intended, the term “one” or similar language is used. Also, as used herein, the terms “has,” “have,” “having,” or the like are intended to be open-ended terms. Further, the phrase “based on” is intended to mean “based, at least in part, on” unless explicitly stated otherwise.

Headings used herein are intended as aids for readability and should not be used to interpret or construe aspects of the technology or claims.

### BRIEF DESCRIPTION OF THE DRAWINGS

FIG. 1. Examples of neurostimulation and neurophysiological recordings.

FIG. 2. Block diagram showing an example of the systems of this disclosure.

FIG. 3. Block diagram showing an example of a technical computing environment of this disclosure.

FIG. 4. Flow diagram of an example process for computing electric and magnetic fields within a high-resolution multi-compartment head model.

FIG. 5. Method of modeling non-nested geometries.

FIG. 6. Distributed dipole method for anisotropic medium.

FIG. 7. Method of improving the convergence using the charge conservation law.

### DETAILED DESCRIPTION OF THE DRAWINGS

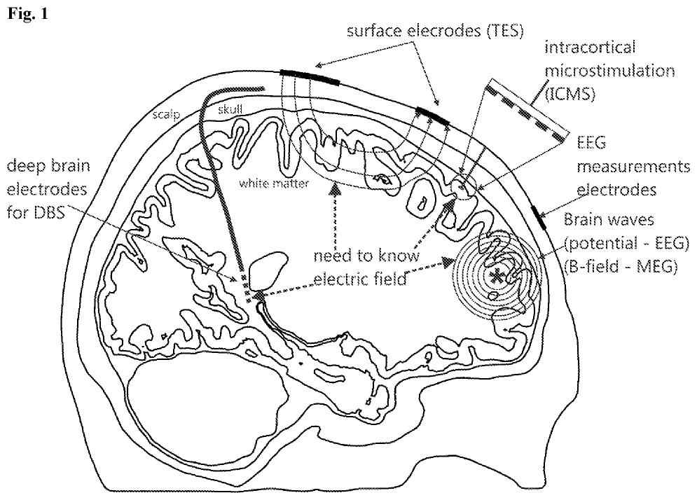

FIG. 1. Examples of TES or transcranial electrical stimulation, DBS or deep brain stimulation, ICMS or intracortical microstimulation and neurophysiological recordings (EEG). and. All applications require fast and accurate computations of the electric field and the electric potential within the head. Neurophysiological recordings additionally include magnetic field measurements (MEG, not shown in FIG. 1) and intracortical EEG (similar to ICMS but with a recording function instead of stimulation).

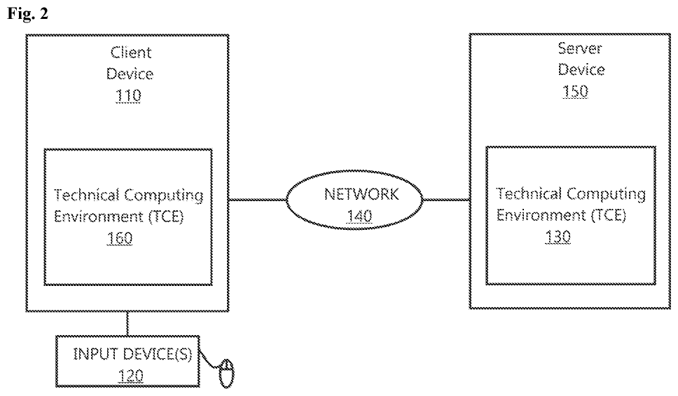

FIG. 2 shows the system example, a client device 110 accesses and interacts with a server device 150 through a network 140 to obtain information about scattering/radiation of a target or choose a target model for use. A user may interact with the client device 110 directly or through one or more input devices 120. The input device(s) 120 can be part of the client device 110 and can be a user device. In some implementations, the client device 110 can provide the desired information without needing to access the server device 150. The server device 150 hosts a technical computing environment (TCE) 130 in which the historical data is processed and the target models are processed and/or generated. In some implementations, the client device 110 may employ a TCE 130 to provide a display, e.g., a webpage. In some implementations, a user interacts with the server device 150 via the displayed webpage. The displayed TCE 130 operating on the client device 110 can be the same as or be part of the TCE 130 hosted by the server device 150.

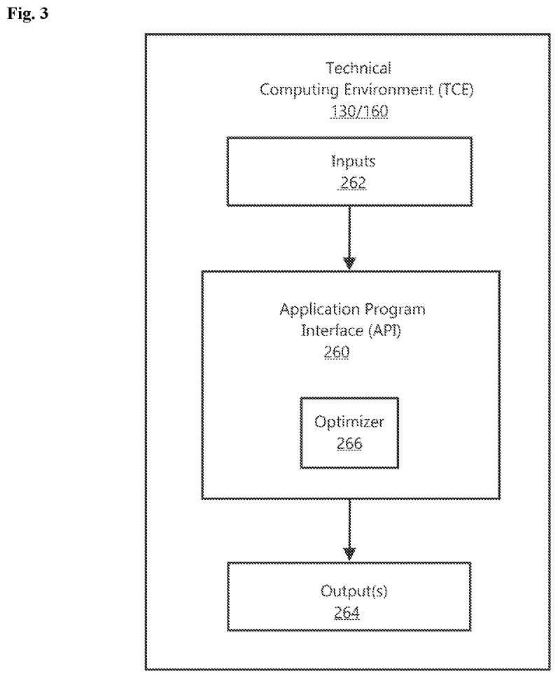

FIG. 3 shows block diagram showing an example of a technical computing environment of this disclosure. The TCE 130 is supported by an application programming interface (API) 260. The API 260 can be visible to a user within the TCE 130, or can be invisible to the user. An example of a portion of programming code is an optimizer 266 that is capable of optimizing parameters. Other examples of the programming code portions can include, but not limited to, a target model generator, equation solver, and graphical user interface (not shown). The API 260 can receive one or more TCE inputs 262 from the TCE 130, or on behalf of a user or a user device, and output one or more TCE outputs 264 to the TCE 130. Examples of TCE inputs 262 include, for example, specifications for the target models to be processed or generated, incident field data, testing data, etc. Examples of TCE outputs 264 include generated models, results of model comparisons, and other user specified information.

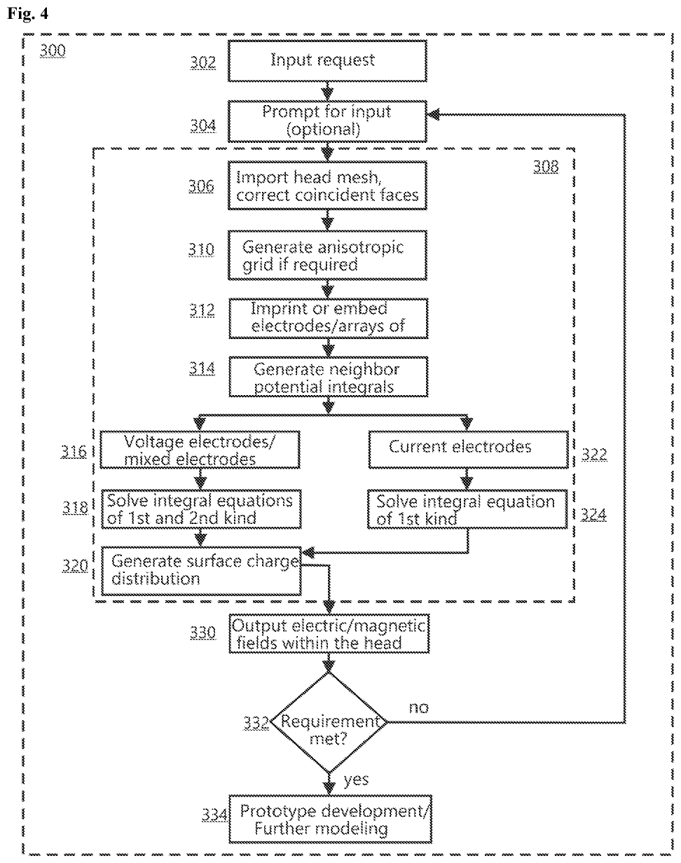

FIG. 4. Flow diagram of an example process for estimating electric and magnetic fields in the brain. The systems of this disclosure can perform process 300 to generate or acquire head target models of the physical subject of interest, and solve for the electric and magnetic fields and other results. Initially, upon receipt of a request, step 302 to perform brain modeling e.g., from a user or a user device, the system may prompt the user or user device to input complete information about the target model(s), step 304. These inputs can be the same as TCE inputs 262 of FIG. 3. Once the target surface head model is imported and described, some implementations then begin to model the fields within the head and performance estimation process (Step 308). Mathematical details of certain exemplary implementations of process 308 are described in much greater detail below as including numerous sub-processes. In step 306, the target head surface mesh model is also processed internally to enable a proper treatment of the non-nested head geometry. In Step 310, a uniform dipole mesh is introduced into an anisotropic head compartment. In Step 312, the electrodes with known parameters are either imprinted or embedded automatically and their mesh labels are generated. In Step 314, neighbor potential integrals necessary for accurate integration are computed as described below. In steps 316, 318, 320, 322, 324, the resulting system of BEM equations is solved iteratively using fast multipole method (FMM) acceleration and generalized minimum residual method (GMRES). Results, such as described above, of the electric and magnetic field estimation process 308 may be output in Step 330. A determination (Step 332) may then be made to establish whether the determined performance characteristics of the electrode system and its readings meet user's requirements. If the requirements are satisfied, the process may then proceed to other target modeling tasks, or progress to prototype development (Step 334). If the performance measures output does not meet the specified requirements, the process may iterate, e.g., from step 332, permitting the user to enter additional and/or different input parameters or change the electrode assembly.

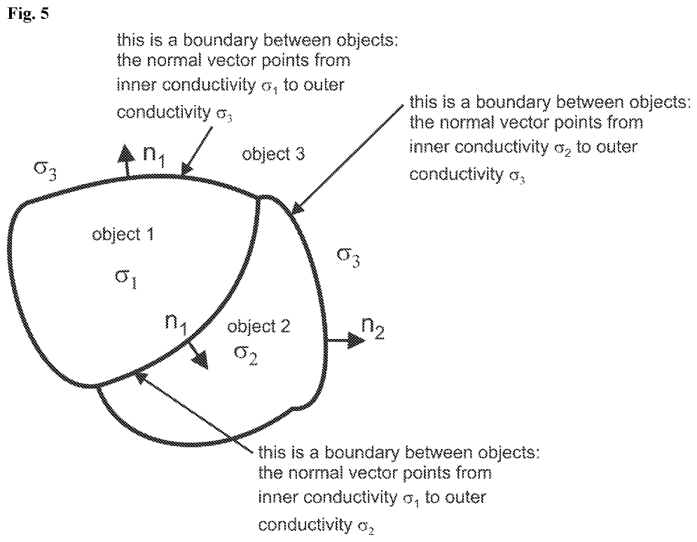

FIG. 5. For two objects (1 and 2) in contact with each other, the joint interface between them should be counted only once. In the figure, this interface is counted only for object 1 with σ₁ being the inner conductivity and σ₂ being the outer conductivity (in the direction of the normal vector n₁). Facets of object 2 at the interface are removed to avoid double-counting.

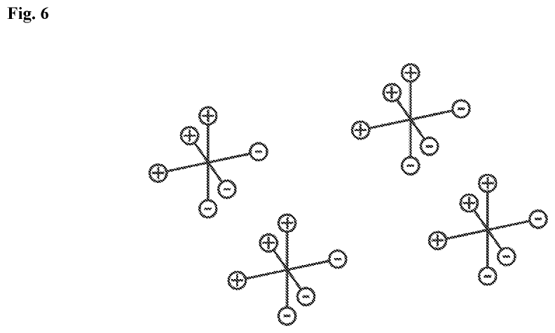

FIG. 6. Model of elementary dipoles uniformly distributed in space used to for taking into account the effect of the anisotropic medium. Three orthogonal dipoles are responsible for three different conductivity values in the x, y, and z directions.

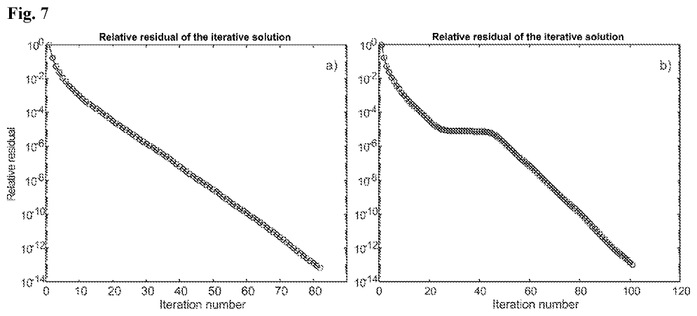

FIG. 7. Typical convergence rate of the solution with generalized minimum residual method (GMRES) for a multi-compartment head model with the charge conservation law; b)—the same result when the charge conservation law is ignored.

1. A method for making a new BEM-FMM computational approach applicable to practical electromagnetic brain modeling for clinical applications including both neurophysiological recordings and neuro stimulation, comprising:
Solving a first integral equation, Eq. (4), and a second integral equation, Eq. (7), for surface charge density ρ(r) residing on tissue conductivity interfaces within a human head model with the new BEM-FMM computational approach,
wherein a tissue conductivity interface is a boundary between any two distinct tissues from a plurality of cranial and intracranial tissues,
wherein the first integral equation, Eq. (4), on the tissue conductivity interfaces is discretized into a plurality of pulse bases on a plurality of triangular facets as shown in a first discretizing equation, Eq. (8a),
wherein the second integral equation, Eq. (7), on an electrode surface is discretized into a plurality of pulse bases on a plurality of triangular facets as shown in a second discretizing equation, Eq. (8b),
wherein a special diagonal preconditioner for the second discretizing equation, Eq. (8b), on an electrode interface in the form of an equation, Eq. (9), as an inverse integral electric potential of a uniform charge distribution on a surface of a given facet, is applied,
wherein the resulting system of coupled equations, Eq. (8a) and Eq. (8b), is solved using a standard fast multipole method and a plurality of iterative methods including a generalized residual method and a bi-conjugate gradient method,
wherein a set of neighbor double surface potential integrals in the form of an Eq. (6) and an Eq. (10) are precomputed analytically,
Inclusion and modeling at least one non-nested human head topology,
Inclusion and modeling at least one anisotropic tissue of the head,
Inclusion and modeling at least one external and at least one internal voltage electrode,
Inclusion and modeling at least one external and at least one internal current electrode,
Improving the convergence of an iterative solution using a charge conservation law,
Restoring at least one near-surface normal electric field from a computed charge distribution without postprocessing.

2. The method of claim 1 wherein the inclusion and modelling of the at least one non-nested head topology further comprises:
Defining non-nested head topology as such where an object 1 and an object 2 may have a joint interface,
Counting the joint interface between the object 1 and the object 2 only once,
Assigning conductivity of the object 1 as an inner conductivity and assigning conductivity of the object 2 as an outer conductivity in a direction of an outer normal vector of object 1,
Interchanging a meaning of objects 1 and 2 if necessary,
Solving the first integral equation, Eq. (4), and the second integral equation, Eq. (7), for the surface charge density ρ(r) residing on conductivity interfaces within a human head model with the new BEM-FMM computational approach as described in claim 1,
Applying this method to a plurality of non-nested head compartments.

3. The method of claim 1 wherein the inclusion and modeling at least one anisotropic tissue further comprises:
Defining an anisotropic tissue as such where a local electrical conductivity and a local dielectric constant are different in different directions,
Defining an elementary dipole source as combination of an electric current source and an electric current sink of equal strength, located closely to each other in a three-dimensional space,
Replacing an anisotropic medium by a volumetric density of such dipole sources uniformly distributed in the three-dimensional space, wherein the space further comprises an x-direction, a y-direction and a z-direction in Cartesian coordinates,
wherein local volumetric dipole density in the x-direction is proportional to (σₓ−σ_{ref})/(σₓ+σ_{ref}) where σₓ is the electrical conductivity in the x-direction, and σ_{ref} is a reference conductivity, and is further proportional to an applied electric field,
wherein local volumetric dipole density in the y-direction is proportional to (σ_{y}−σ_{ref})/(σ_{y}+σ_{ref}) where σ_{y} is the electrical conductivity in the y-direction, and σ_{ref} is a reference conductivity, and is further proportional to an applied electric field,
wherein local volumetric dipole density in the z-direction is proportional to (σ_{z}−σ_{ref})/(σ_{z}+σ_{ref}) where σ_{z} is the electrical conductivity in the z-direction, and σ_{ref} is a reference conductivity, and is further proportional to an applied electric field,
Solving the first integral equation, Eq. (4), and the second integral equation, Eq. (7), for the surface charge density ρ(r) residing on conductivity interfaces within a human head model with the new BEM-FMM computational approach as described in claim 1,
wherein the primary field E^{p}(r) on the right hand side of the first integral equation, Eq. (4), now contains the field of the said volumetric density of dipole sources.

4. The method of claim 1 wherein inclusion and modeling at least one external and at least one internal voltage electrode further comprises,
Defining an external voltage electrode as such located on a skin surface and assigned a certain voltage V,
Defining an internal voltage electrode as such located within the human head model and assigned a certain voltage V,
Solving the first integral equation, Eq. (4), and the second integral equation, Eq. (7), for the surface charge density ρ(r) residing on conductivity interfaces within the human head model with the new BEM-FMM computational approach as described in claim 1.

5. The method of claim 1 for inclusion and modeling an external current electrode and an internal current electrode further comprises,
Defining an external current electrode as such located on a skin surface and assigned a certain current Iₑ,
Defining an internal current electrode as such located within the human head model and assigned a certain current Iₑ,
Setting a primary electric field E^{p}(r) in the first integral equation, Eq. (4), in the form n(r)·E^{p}(r)=±Iₑ(σₑAₑ),
wherein the electrode has a local normal vector n(r) and a certain electrode area Aₑ,
wherein the electrode is enclosed in a medium with conductivity σₑ for the internal electrode,
wherein σₑ is a scalp conductivity for the external electrode.
Solving only the first integral equation, Eq. (4), for the surface charge density ρ(r) residing on conductivity interfaces within a human head model with the new BEM-FMM computational approach as described in claim 1.

6. The method of claim 1 further comprising,
Adding a charge conservation law, Eq. (11), to the first integral equation, Eq. (4), with a certain weight value,
wherein S in Eq. (11) is a combination of all interfaces in the first integral equation, Eq. (4),
wherein a weight value on the order of 1 is assigned to Eq. (11), more preferably the weight value is one half,
Solving only the first integral equation, Eq. (4), for the surface charge density ρ(r) residing on conductivity interfaces within a human head model with the new BEM-FMM computational approach as described in claim 1.

7. The method of claim 1 further comprising,
Using an algebraic equation, Eq. (12) which directly expresses a normal electric field in terms of local surface charge density ρ(r) residing on tissue conductivity interfaces and two interfacial conductivities σᵢₙ and σₒᵤₜ,
wherein the normal electric field is the normal field just inside any tissue conductivity interface and just outside any tissue conductivity interface, and the normal field discontinuity across an interface,
wherein the surface charge density ρ(r) residing on conductivity interfaces within a human head model is found with the new BEM-FMM computational approach as described in claim 1.

8. The method of claim 1 further comprising,
Including a dielectric permittivity ε of a human tissue into consideration by replacing the two interfacial conductivity values in the first integral equation, Eq. (4), by a complex conductivity in the form of an equation σ_{in/out}→σ_{in/out}+jωε_{in/out} for a harmonic excitation with an angular frequency ω where the dielectric permittivity ε is a relative dielectric constant of the tissue,
Solving the first integral equation, Eq. (4), and the second integral equation, Eq. (7), for the surface charge density ρ(r) residing on conductivity and dielectric interfaces within a human head model with the new BEM-FMM computational approach as described in claim 1.

9. The method of claim 1 further comprising,
Defining a primary field E^{p}(r) on the right hand side of the first integral equation, Eq. (4), through a magnetic vector potential, A, of a transcranial magnetic stimulation (TMS) coil in the form E^{p}(r)=−∂A/∂tA, without a current or at least one voltage electrode,
Solving only the first integral equation, Eq. (4), for the surface charge density ρ(r) residing on conductivity interfaces within a human head model with the new BEM-FMM computational approach as described in claim 1.

10. The method of claim 1 further comprising,
A plurality of head models and a plurality of compositions while each may include at least one of the following: a conducting tissue component, a dielectric tissue component, and an anisotropic tissue component.

11. A tangible, non-transitory computer-readable medium storing instructions, the instructions when read by one or more processors, cause the one or more processors to:
Acquire the human head model with a plurality of head compartments and a plurality of tissue properties,
wherein the human head model may have a plurality of shapes and may include at least one of the following: a conducting tissue component, a dielectric tissue component, and an anisotropic tissue component,
Compute an electric and a magnetic field for a plurality of excitations, E^{p}(r), in the first integral equation, Eq. (4), and a plurality of excitations, V, in the second integral equation, Eq. (7), as described in claim 1,
wherein keeping only the excitation in the form E^{p}(r) corresponds to transcranial magnetic stimulation (TMS) modeling,
wherein keeping only the excitation in the form E^{p}(r) also corresponds to electroencephalography (EEG) modeling with a plurality of volumetric current dipole distributions,
wherein keeping only the excitation in the form V corresponds to transcranial electrical stimulation (TES) or deep brain stimulation (DBS) or intracortical micro stimulation (ICMS) modeling.

12. The medium of claim 11, wherein the instructions further cause the one or more processors to optimize performance of a medical diagnostic tool.

13. A device comprising one or more processors configured to:
Acquire the human head model with a plurality of head compartments and a plurality of tissue properties,
wherein the human head model may have a plurality of shapes and may include at least one of the following: a conducting tissue component, a dielectric tissue component, and an anisotropic tissue component,
Compute an electric and a magnetic field for a plurality of excitations, E^{p}(r), in the first integral equation, Eq. (4), and a plurality of excitations, V, in the second integral equation, Eq. (7), as described in claim 1,
wherein keeping only the excitation in the form E^{p}(r) corresponds to transcranial magnetic stimulation (TMS) modeling,
wherein keeping only the excitation in the form E^{p}(r) also corresponds to electroencephalography (EEG) modeling with a plurality of volumetric current dipole distributions,
wherein keeping only the excitation in the form V corresponds to transcranial electrical stimulation (TES) or deep brain stimulation (DBS) or intracortical micro stimulation (ICMS) modeling.
